(19) United States
(12) Patent Application Publication
(10) Pub. No.: US 2012/0082742 A1
(43) Pub. Date: Apr. 5, 2012

(54) METHOD AND COMPOSITIONS FOR TREATING CUTANEOUS VERRUCAE

(75) Inventor: Joel E. Bernstein, Deerfield, IL (US)

(73) Assignee: ELORAC, LTD, Vernon Hills, IL (US)

(21) Appl. No.: 12/895,070

(22) Filed: Sep. 30, 2010

(51) Int. Cl. A61K 36/15; A61P 31/00; A61K 31/341; A61P 17/00; A61K 31/60; A61K 31/19

(52) U.S. Cl. 424/770; 514/159; 514/557; 514/474

(57) ABSTRACT

By incorporating certain pharmaceutical and cosmetic preservative agents into nail polish vehicles containing corrosive ingredients, the risk and prevalence of recurrence of verrucae can be substantially reduced.

### BACKGROUND

Verrucae or warts are benign intraepidermal growths of the skin caused by viruses of the human papilloma virus (“HPV”) family. Verrucae commonly appear in children and young adults on the hands, face and feet. HPA virions are small double-stranded DNA viruses which, unlike herpes viruses, do not possess a lipoprotein envelope. HPV is acquired through breaks in the skin. Once infected, new warts may develop over a small area or in distant sites by autoinoculation.

The relative abundance of virions in a wart varies with the immunological status of the host, the HPV type, and the location of the lesions. New verrucae usually contain more virions than do older verrucae. Plantar and palmar warts contain an especially high number of virions.

Treatment of verrucae commonly employs the use of agents with sufficient corrosive activity so as to cause peeling and eventual removal of the hyperkeratotic lesions. Agents used in this manner include salicylic acid, lactic acid, acetic acid, trichloroacetic acid and ascorbic acid. While these agents applied daily for a number of weeks are effective in a good percentage of patients with warts, some have recurrences of verrucae at site of treatment due to persisting HPV virions in the superficial epidermis at the base of the wart.

In U.S. Pat. No. 4,588,590, the inventor disclosed the use of a nail polish vehicle for incorporation of at least one pharmaceutically acceptable corrosive agent sufficient for topical application to the lesion to cause peeling of keratotic lesions including warts. Although these compositions and the method of treatment provided a significant improvement in the ease of treatment of warts, unfortunately the risk and prevalence of recurrence of the wart at the treatment site was not positively affected. This failure to reduce recurrence rates is likely due to HPV virions remaining in the superficial epidermis below the wart base.

### SUMMARY

By incorporating certain pharmaceutical and cosmetic preservative agents into nail polish vehicles containing corrosive ingredients, the risk and prevalence of recurrence of verrucae can be substantially reduced.

Methods and compositions for treating verrucae which reduce recurrence of verrucae at sites of treatment are herein described.

Compositions for treatment of verrucae are disclosed that include a nail polish vehicle containing an effective amount (about 5% to 40% by weight) of at least one pharmaceutically acceptable corrosive agent sufficient to cause peeling of the verrucae, in combination with preservative systems which contain at least one of certain pharmaceutical and cosmetic preservative agents which have, in addition to their antibacterial and/or antifungal activity, antiviral activity against the HPV. Suitable preservative agents include behenic alcohol (aka docosyl alcohol), phenol, cresol, thymol, resorcinol, pine tar and pine oil. Combinations of low dosages (dosages≦2% of each preservative agent) of these preservative agents are equally or more effective at reducing verrucae recurrence rates than high doses (dosages>5% of a preservative agent) of a single preservative agent.

### DETAILED DESCRIPTION

It has been discovered surprisingly that incorporating certain pharmaceutical and cosmetic preservative agents, which have antiviral activity, together with antibacterial and/or antifungal activity, into nail polish vehicles containing corrosive agents, successfully treats warts, while producing a lower risk and prevalence of recurrence of the lesions at treatment sites.

One or more corrosive agents selected from a group that includes salicylic acid, lactic acid, acetic acid, trichloroacetic acid and ascorbic acid, are incorporated into a nail polish vehicle having a preservative system containing one, or preferably more than one, of the following preservatives which have antiviral activity: behenic alcohol, phenol, cresol, thymol, resorcinol, pine tar, and pine oil. Although the corrosive agents are incorporated into the nail polish vehicles in amounts between about 5% and 40% by weight, the preservative agents are incorporated into the resulting formulations in amounts between about 0.05% and 10% by weight.

Low dosages of combinations of two or more of the preservatives with antiviral activity specified herein are preferred over high doses of a single preservative agent. Thus a combination of 0.2% phenol and 0.2% thymol by weight is as effective in reducing wart recurrences as 1.0% by weight of phenol or thymol individually.

### EXAMPLES

### Example 1

Salicylic acid 28% by weight was incorporated into a nail polish vehicle containing by weight 60% isopropyl alcohol, 27% butyl acetate, 0.3% acrylates copolymer, 10% butyl acetate, 2% dibutyl phthalate, 0.4% phenol and 0.3% thymol. The resulting lotion is applied once daily to the wart surface for 2 weeks with complete resolution of the wart. Four weeks after disappearance of the wart, there is no sign of recurrence at the treatment site.

### Example 2

A nail polish composition was prepared by mixing 25% by weight salicylic acid in a polish vehicle containing by weight 65% isopropyl alcohol, 21% butyl acetate, 9.5% polyvinyl butyral, 3.0% trimethyl pentanyl dissobutyrate, 0.5% monohydric caprolactone monoacrylate, 0.5% phenol, 0.2% thymol, and 0.3% behenic alcohol. The resulting composition when applied daily to the surface of a wart dissolves the proliferative keratinous wart, while destroying the HPV virions at the base of the wart, resulting in both a rapid disappearance of the wart with little or no risk of recurrence.

### Example 3

The nail polish vehicle of Example 1 with a combination by weight of 15% glacial acetic acid and13% lactic acid replacing the 28% salicylic acid of Example 1.

### Example 4

Trichloracetic acid 10% by weight and 5% ascorbic acid by weight are incorporated into a nail polish vehicle composed by weight of 58% isopropyl alcohol, 20% butyl acetate, 10% polyvinyl butyral, 3% trimethyl pentanyl dissobutyrate, 0.3% acrylates copolymer, 4.0% phenol, 4.0% cresol, and 0.7% pine oil. The resulting nail polish composition is applied once daily to the surface of the wart for 3 weeks with the resulting disappearance of the wart and elimination of HPV virions from the affected site.

### Example 5

Salicylic acid 40% by weight is mixed into a nail polish vehicle composed by weight of 72% isopropyl alcohol, 10% butyl acetate, 8% polyvinyl butyral, 2.5% trimethyl pentanyl dissobutyrate, 0.5% acrylates polymer, 5% behenic alcohol, 1% phenol, 0.5% resorcinol, and 0.5% pine tar. The resulting nail polish composition, which combines the strong caustic activity of 40% salicylic acid with the antiviral activity of its preservative complex composed of 5% behenic alcohol, 1% phenol, 0.5% resorcinol and 0.5% pine tar, provides prompt resolution of the wart through the keratolytic activity of salicylic acid with substantially reduced risk of recurrence at the site through the antiviral activity of its preservative complex.

1. A method of treating cutaneous verrucae (warts), the method comprising daily applying to the wart a nail polish composition containing an effective amount of at least one pharmaceutically acceptable corrosive agent sufficient to cause peeling of the wart, as well as containing one or more preservative agents which possess antiviral activity in addition to their antibacterial and/or antifungal activity in a concentration sufficient to kill virions within the wart.

2. A method as in claim 1, wherein the corrosive agent is selected from the group consisting of salicylic acid, lactic acid, acetic acid, trichloroacetic acid and ascorbic acid.

3. A method as in claim 1, wherein the concentration of the corrosive agent or agents in the nail polish vehicle is an amount between about 5% and 40% by weight.

4. A method as in claim 1, wherein the one or more antiviral preservative agents are selected from the group consisting of behenic alcohol, phenol, cresol, thymol, resorcinol, pine tar, and pine oil.

5. A method as in claim 1, wherein the total concentration of the antiviral preservative agent or agents is an amount between about 0.05% and 10% by weight.

6. A nail polish composition for treating cutaneous verrucae, the method comprising a nail polish vehicle containing an effective amount of a pharmaceutically acceptable corrosive agent in combination with one or more antiviral preservative agents sufficient to cause peeling of the warts and elimination of wart virions to reduce the incidence of wart recurrence at the site of treatment.

7. A nail polish composition as defined in claim 6, wherein the corrosive agent is selected from the group consisting of salicylic acid, lactic acid, acetic acid, trichloroacetic acid and ascorbic acid.

8. A nail polish composition as defined in claim 6, wherein the total concentration of the corrosive agent or agents is an amount between about 5% and 40% by weight.

9. A nail polish composition as defined in claim 6, wherein the antiviral preservative agent is selected from the group consisting of behenic alcohol, phenol, cresol, thymol, resorcinol, pine tar, and pine oil.

10. A nail polish composition as defined in claim 6, wherein the total concentration of the viral preservative agent or agents is an amount between about 0.05% and 10% by weight.
